(19) United States
(12) Patent Application Publication
(10) Pub. No.: US 2009/0005454 A1
(43) Pub. Date: Jan. 1, 2009

(54) Method of Reducing Nosocomial Infections

(75) Inventor: David Barshis, Scottsdale, AZ (US)

Correspondence Address: 3M INNOVATIVE PROPERTIES COMPANY, PO BOX 33427, ST. PAUL, MN, 55133-3427, US

(73) Assignee: Zila Pharmacceuticals, Inc., Phoenix, AZ (US)

(21) Appl. No.: 10/555,345

(22) PCT Filed: Nov. 1, 2004
(86) PCT No.: PCT/US2004/036362

§ 371 Date: Apr. 16, 2008

(51) Int. Cl. A61K 31/155; A61P 31/00

(52) U.S. Cl. 514/636

(57) ABSTRACT

Nosocomial infections, particularly nosocomial infections of intubated patients, are reduced by applying a liquid disinfectant directly to the mucosa, before intubation, by a swab applicator or by a spray applicator.

This invention relates to methods of reducing nosocomial infections in a mammalian patient.

In another respect the invention concerns such methods which can be used when the patient is unable or unwilling to apply a disinfectant to mucosal tissue by rinsing, gargling or other self-administration methods.

These and other, further and more specific aspects of the invention will be apparent to those skilled in the art from the following description thereof.

### BACKGROUND OF THE INVENTION

Most nosocomial infections are caused by the contamination of medical devices, resulting in serious hospital-acquired infections. Nosocomial pneumonias are the second most common nosocomial infections, and are associated with the highest attributable mortality and morbidity. Recent data have shown that at least 300,000 episodes of nosocomial pneumonia occur annually in the United States. The attributable mortality of this infection is 33%-50%, hence, around 100,000 patients die annually because of nosocomial pneumonia. The risk of nosocomial pneumonia increases six to twenty fold from the use of mechanical ventilation.

The endotracheal tube is considered a common vehicle for colonization/contamination leading to nosocomial pneumonia. The endotracheal tube connects the oropharyngeal environment with the sterile bronchoalveolar space, significantly increasing the risk of nosocomial pneumonia.

Other sites, which may invaded by harmful bacteria, causing nosocomial infections, include the inner ear and the nasal passages.

### THE PRIOR ART

Prior attempts to reduce the incidence of nosocomial infections have proposed methods for sterilizing devices by application of disinfectant coatings to the surfaces of the devices (US 20030078242 A1) and by sterilizing peripheral apparatus used in surgery such as storage drawers (US 20040135967 A1), surgical gloves (US 20040151919 A1) and the like. Other methods involve use of topical disinfectants applied to the surfaces of the oral and oropharyngeal cavity before intubation by the patient gargling a liquid containing the disinfectant, e.g, chlorhexadine gluconate. (Houston et al., Am. J. Crit. Care, November 2002, 11 No. 6).

Despite the marked reduction in nosocomial pneumonia in intubated patients by having the patient gargle a liquid disinfectant, there is a significant problem in applying a disinfectant when the patient is either unwilling or unable to gargle the disinfectant or take other self-application actions.

### BRIEF DESCRIPTION OF THE INVENTION

Briefly, I provide a method of reducing nosocomial infections of a mammalian patient who is unwilling or unable to self-apply a disinfectant, by applying a disinfectant to the patient's mucosa by spraying the disinfectant or by use of a swab carrying the disinfectant.

In the presently preferred embodiment of the invention, the disinfectant is a solution of a chlorhexadine derivative, most preferably chlorhexadine gluconate.

### BRIEF DESCRIPTION OF THE DRAWINGS

FIG. 1 depicts a swab applicator useful in practicing the preferred method of the invention for disinfecting the oropharyngeal cavity;

FIG. 2 is a sectional view of the swab of FIG. 1, taken along section line 2-2 thereof;

FIG. 3 is a perspective view of a spray applicator suitable for use in practicing the invention.

### DETAILED DESCRIPTION OF THE INVENTION

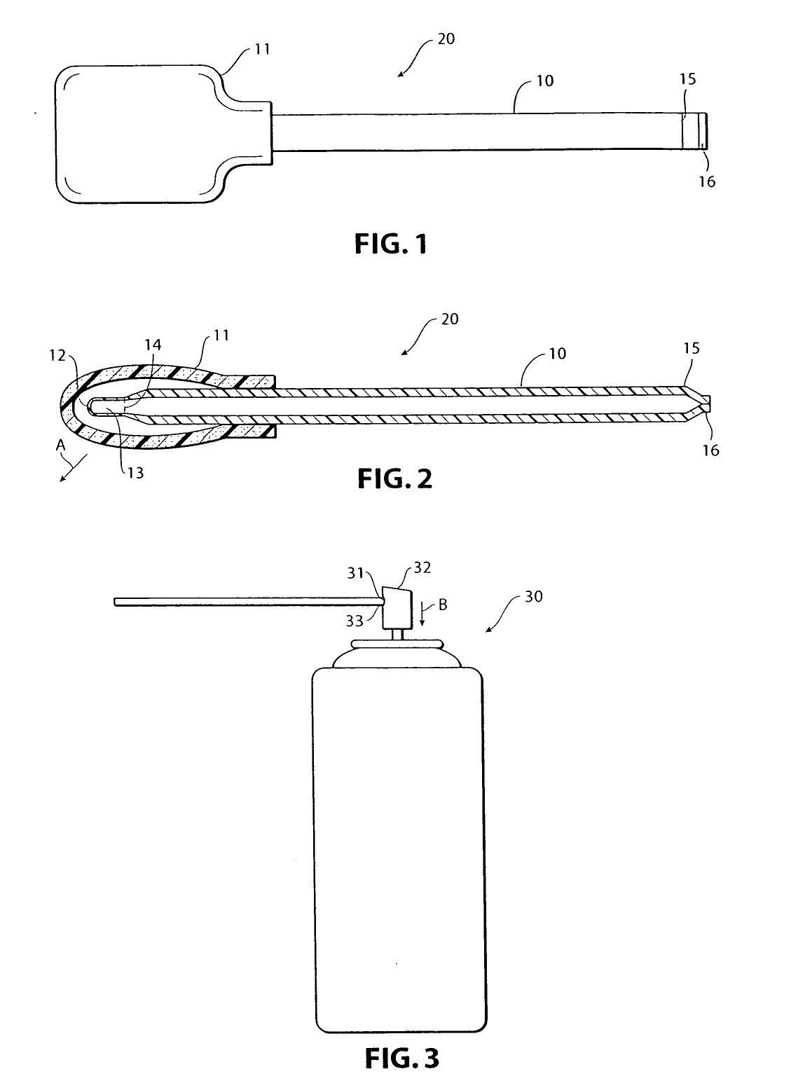

FIGS. 1 and 2, depict a swab applicator, generally indicated by reference numeral 20, useful in practicing a preferred embodiment of the method of the invention and in which like reference characters identify the same elements in the several views. The swab applicator 20 consists of a hollow cylindrical handle portion 10 carrying a generally flat shaped porous sponge 11 on the distal end 12 of the handle 10. The distal end 12 is formed as a frangible tip 13 inside the sponge 11, which can be opened by pressing downwardly and outwardly in the direction of the arrow A to cause the tip 13 to separate from the distal end 12 of the handle 10 at the score line 14. The hollow handle 10 is filled with a liquid disinfectant and the upper end 15 of the handle 10 is then closed by a heat seal 16.

As depicted in FIG. 2, the distal edges 21 of the sponge 11 are tapered inwardly on the sides 22 and on the distal end 23 of the sponge 11, to facilitate manipulation of the swab into narrow spaces in the oral and oropharyngeal cavities, e.g., between the patient's cheek and gums. For adult human patients, the flattened sponge 11 is approximately one inch wide and long and approximately ¼ inch thick. For applying a liquid disinfectant to other mucosal surfaces such as the nasal passages or in the ear, the shape of the sponge is modified to adapt the swab to apply the disinfectant within these narrower passages.

FIG. 3 depicts a spray applicator can 30 which is filled with the liquid disinfectant (not shown), provided with an atomizing valve 31 which is actuated by pressing downwardly in the direction of the arrow B. The spray of disinfectant is expelled through the port 31 in the valve 32 by either pressurizing the can 30 with a non-toxic gas or by using a pump-type atomizing valve 32. To assist in directing the disinfectant liquid to mucosal areas, such as the oral and oralpharyngeal surfaces, the nasal cavity or the ear, a cylindrical pipe 32 is provided, the proximal end 33 thereof having a outside diameter sized to be inserted into the port 31, so that, when so inserted, the disinfectant liquid is sprayed from the distal end 33 of the pipe 32.

The swab applicator 20 and the spray applicator 30 can be used alone or in combination to dispense and apply the disinfectant liquid as contemplated by my invention. To facilitate application of the disinfectant and retention of it on mucosal surfaces, the viscosity of a liquid solution of the disinfectant can be adjusted by incorporating viscosity modifiers such as thickeners, e.g., hydroxypropyl cellulose, surfactants, and the like. Depending on the patient and the location of the mucosa to be treated, the viscosity of the disinfectant can be varied from a very low viscosity alcohol solution to a semi-solid mousse.

The method of my invention is also applicable to veterinary practice, in which case it may be desirable to increase or decrease the size and/or shape of the swab applicator or spray applicator and adjust the viscosity of the disinfectant appropriately.

The following examples are presented in order to enable persons skilled in the art to understand and practice my invention and to indicate the presently preferred embodiments thereof. These illustrative examples are not intended to indicate the scope of the invention, which is defined only by the appended claims.

### EXAMPLE 1

The swab applicator of FIGS. 1-2 is used to apply an alcoholic chlorhexadine gluconate disinfectant liquid to a human patient who has been anesthetized prior to surgery which will require intubation. This disinfectant is commercially available under the registered trademark PERIDEX® from Zila Pharmaceuticals, Inc. of Phoenix, Ariz., USA.

Before intubation, the handle of the swab applicator is pressed downwardly and sideways to separate the frangible tip at the score line of the handle, allowing the disinfectant liquid to flow into and saturate the sponge.

The disinfectant liquid is liberally applied with the swab to the attached gingiva, the buccal mucosa, the floor of the mouth, the hard and soft palate and the dorsal, lateral and ventral tongue and to the exposed oropharyngeal surfaces. The patient is then intubated with a sterile endotracheal tube.

### EXAMPLE 2

The procedure of Example 1 is repeated except that a pressurized spray applicator of FIG. 3 is employed instead of the swab applicator of Example 1, to apply the solution of Example 1 to the patient's nasal passage mucosa.

Having described by invention in such terms as to enable those skilled in the art to understand and practice it and, having identified the presently preferred embodiments thereof,

I claim:

1. The method of reducing nosocomial infections of a mammalian patient who is unwilling or unable to self-apply a disinfectant, comprising applying a liquid disinfectant to the patient's mucosa by spraying the disinfectant or by use of a swab carrying the disinfectant.

2. The method of claim, 1 wherein said liquid disinfectant is applied by spraying.

3. The method of claim 1, wherein said liquid disinfectant is applied by use of a swab carrying the disinfectant.

4. The method of claim 1, wherein said liquid disinfectant comprises a chlorhexadine derivative.

5. The method of claim 1, wherein said derivative is chlorhexadine gluconate.

6. The use of chlorhexadine gluconate in formulating a disinfectant for use according to the method of claim 1.

7. A method of preventing nosocomial infections, comprising:
applying a disinfectant to an area of the patient's mucosa, wherein the disinfectant comprises chlorhexadine gluconate;
inserting an endotracheal tube into the patient's trachea such that it is in contact with the area of the patient's mucosa.

8. The method of claim 7, wherein the area of the patient's mucosa is at least one selected from the group consisting of the gingiva, the buccal mucosa, the floor of the mouth, the hard palate, the soft palate, the dorsal tongue, the lateral tongue, the ventral tongue, and an oropharyngeal surface.

9. The method of claim 7, wherein the step of applying a disinfectant comprises contacting a swab applicator with the disinfectant and contacting the area of the patient's mucosa with the swab applicator.

10. The method of claim 7, further comprising providing a spray can containing the disinfectant, and wherein the step of applying a disinfectant comprises spraying the disinfectant on the area of the patient's mucosa.

11. A method of preventing nosocomial infections, comprising:
providing a swab applicator comprising a handle having a frangible tip and a sponge disposed about the frangible tip, wherein the handle contains a disinfectant comprising chlorhexadine gluconate;
separating at least a portion of the tip from the handle such that the disinfectant contacts the sponge; and
contacting an area of the patient's mucosa with the sponge.

12. The method of claim 11, wherein the frangible tip is scored.

13. The method of claim 11, further comprising inserting an endotracheal tube into the patient's trachea such that is in contact with the area of the patient's mucosa.

14. The method of claim 11, wherein the area of the patient's mucosa is at least one selected from the group consisting of the gingiva, the buccal mucosa, the floor of the mouth, the hard palate, the soft palate, the dorsal tongue, the lateral tongue, the ventral tongue, and an oropharyngeal surface.
